(19) United States
(12) Patent Application Publication
(10) Pub. No.: US 2020/0376205 A1
(43) Pub. Date: Dec. 3, 2020

(54) DOSAGE DETERMINATION USING ELECTRICAL CONDUCTIVITY IN AN INJECTION DEVICE

(71) Applicant: Haselmeier AG, St. Gallen (CH)

(72) Inventor: Joachim Keitel, Esslingen (DE)

(21) Appl. No.: 16/996,533

(22) Filed: Aug. 18, 2020

Related U.S. Application Data
(63) Continuation of application No. PCT/EP2019/053943, filed on Feb. 18, 2019.
(60) Provisional application No. 62/635,386, filed on Feb. 26, 2018.

(51) Int. Cl. A61M 5/315; A61M 5/24

(52) U.S. Cl. CPC A61M 5/31568; A61M 5/31551; A61M 5/31581; A61M 2005/2485; A61M 5/31573; A61M 2205/3317; A61M 2205/0233; A61M 5/24

(57) ABSTRACT

A dose setting mechanism for a drug delivery device is presented that includes one or more potentiometers whereby relative motion and distances traveled between components in the dose setting mechanism are determined by monitoring and measuring changes in electrical resistance using a measuring device in electrical communication with the one or more potentiometers.

### CROSS REFERENCE TO RELATED APPLICATIONS

This application is a continuation application of international patent application PCT/EP2019/053943, filed Feb. 18, 2019, designating the United States and claiming priority from U.S. provisional application 62/635,386, filed Feb. 26, 2018, and the entire content of both applications is incorporated herein by reference.

### TECHNICAL AREA

The present disclosure relates to an injection device and particularly to the dose setting mechanism of the injection device, where a dose of medicament is measured using a linear potentiometer. A set dose, a corrected dose and/or a delivered dose can be determined by measuring electrical conductivity of a circuit having one or more conductive tracks positioned within the injection device.

### BACKGROUND

There are a number of medicament delivery devices on the market that are capable of automatically, semi-automatically or manually delivering doses of medicament. Of the known type of delivery devices is the “pen-type” injector which is popular and is available in both reusable and disposable designs. Such devices are constructed with dose setting mechanisms that include a variety of inter-acting mechanical components to achieve desired functions, such as setting a dose, dose cancellation, and ultimately delivering the set dose. Such devices are typically designed for non-medically trained individuals to self-administer medicaments. Users of such devices include diabetic patients, where medication management and compliance, i.e. the degree to which a patient follows medical instructions and protocols, is often of extreme importance. To evaluate and determine compliance of a self-medicating user, it is desirable to obtain as much information about each injection as possible, for example, the determination of the actual dose of the medication injected, the amount of the set dose, whether a dose setting correction was needed, the rate of dose injection, whether the injection was halted, the time of day when the injection was performed, and the time required to complete the injection. Collection and evaluation of such data can be especially important if the user is physically impaired, for example, having reduced eyesight, lacking of cognitive skills, or severe arthritis.

With the need to collect and evaluate the above-identified injection parameters as goal of health care providers, it is desirable to provide medication delivery systems that are economical to manufacture and that can monitor and record injection activities or that are ready and capable of working with other devices to monitor, record and report user compliance with injection protocols. As such, it an object of the present disclosure to provide an injection device, preferably a disposable device, that is manufactured in a “ready state” to allow the above-mentioned injection parameters to be measured, recorded and transmitted so that the collected user data can be evaluated by one or more health care professionals. The “ready state” injection device would be disposable and designed to provide electrical connectors for attachment of a reusable measuring device that can monitor, measure, collect and compute electrical conductivity data from the injection device each time a user handles or otherwise performs an injection, thus allowing for cost effective manufacturing of reusable and disposable injection devices.

The disclosure presented below achieves the above-mentioned goals by providing a injection device design that allows for the evaluation of user compliance with medical treatment protocols.

### BRIEF DESCRIPTION

This disclosure is applicable to a number of dose setting mechanism designs for use in drug delivery devices, for example, in pen-type injectors, that allow for the monitoring of the injection parameters used to deliver doses of medicaments. Each of the possible designs disclosed herein is generally based on the working principle of a potentiometer, preferably a linear potentiometer. In its broadest sense the injection devices of this disclosure employ one or more tracks or strips of conductive material having a certain electrical resistance. A wiper, also made of conducting material, is in electrical contact with the track and is designed such that the wiper moves relative to track or vice versa, i.e., the track moves relative to the wiper that is being held in a fixed position.

By applying a fixed voltage with known current across one contact connected to the wiper and to a second contact located on one end of the track, the electrical conductivity or resistance can be measured and determined using Ohm's law, which states that the current through a conductor between two points is directly proportional to voltage across two points. Stated differently, resistance (R) is equal to voltage (V) divided by current (I) or R=V/I. Further, because the resistance of the conductor is proportional to the length of the track of the conductive material, as the wiper moves relative to the track the resistance measured will vary, e.g., as the distance between the contact on the wiper and the contact at the end of the track gets shorter, the measured resistance is less. Likewise, the greater the distance along the track to the wiper, the higher the resistance. This measured electrical conductivity is also directly proportional to the relative movement of two component parts in the injection device, which in turn can be directly correlated to a dose of medicament set by the user and/or a dose of medicament delivered during the injection procedure.

Preferably, the device of the present disclosure is a pen-type injection device that is capable of variable, user settable, multiple doses from a single container of medicament, where the container is preferably a cartridge. Examples of such devices are described in U.S. Pat. No. 8,512,296, and U.S. Pub. No. 2018/0001031, the contents of each of these patent disclosures are fully incorporated by reference in this application. The injection device can be reusable, meaning that the container of medicament is replaceable through partial disassembly and resetting of the injection device, for example by replacing an empty cartridge with a full cartridge and retracting a piston rod back into a dose setting mechanism. In a reusable device, a cartridge holder is removed from the proximal end of the dose setting mechanism and the old empty cartridge is replaced by a new full cartridge and the cartridge holder is reattached to the dose setting mechanism. In a disposable injection device, the cartridge holder is permanently attached to the dose setting mechanism and once the cartridge of medicament is empty, the entire injection device is disposed of.

The present potentiometric determination of dosage as described in the present disclosure is applicable to a wide variety of injection device designs, provided that at least one component of the dose setting mechanism moves linearly during dose setting, dose correction, and dose delivery. This linear movement can be, but not always be, directly proportional to an amount of medicament that would be expelled from the container of medicament if the injection procedure was fully carried out. Two possible dose setting components that move linearly are described in more detail below. One such component is a piston rod and other is a dose sleeve that translates relative to the device outer housing in a distal direction during dose setting and translates proximally relative to the outer housing during dose delivery or a dose setting correction procedure.

The determination of changing electrical conductivity or resistance is preferably performed using at least one track of conductive material that is applied to or integral with a component of the dose setting mechanism that moves longitudinally during dose setting, dose correction and dose delivery. A wiper is positioned on or integral with another component of the injection device so that the wiper is in contact with the track and is linearly fixed relative to the track. Measuring the electrical resistance between the wiper and the track during the relative movement of the two device components will be a direct measurement of the physical and relative position of the two device components.

A most preferred and efficient way to measure the resistance is to use two longitudinal conductive tracks where the wiper is in contact with both tracks. In this manner electrical connectors are located at same ends of each track. Such a design will work when the tracks move relative to the wiper or vice versa. Contacting the two tracks via a conductive strip will work when two non-connected wipers are used, with one wiper in sliding contact with one track and the other wiper in sliding contact with the other track, regardless of whether the tracks move relative to the wipers or vice versa. These electrical connectors at the ends of the tracks or the wipers are positioned on the injection device so that they are accessible by a separate measuring device that monitors, measures, collects, computes and records data relating to dose setting, dose corrections, and dose delivery. Preferably the separate measuring device is attachable, removable and reusable relative to the injection device of the present disclosure. By having a separate and reusable measuring device allows the injection device to be manufactured economically in a “ready state”, meaning ready for attachment of the measuring device. Further details of the separate measuring device will be disclosed below.

The conductive track(s) can be made from conductive plastic strips having a surface with an electrical resistance which is proportional to the length of the strip. Alternatively, the conductive track or wiper made be fabricated using conductive paint, ink, or other conductive medium that can be placed on or incorporated within the selected dose setting components. Regardless of the method of manufacture, it is preferred to dope (embed) the composition with conductive material such that it has the appropriate resistance characteristic desired. Effectively, the track, e.g., conductive plastic strip, is a resistor which can be “tapped” at any point on its surface by the wiper(s). The longer the distance between any two tap points, the higher the resistance. A single wiper is the component that taps the conductive track and provides one electrical connection for determining resistance or changing resistance during relative movement between the track(s) and wiper. The wiper(s) can be made of the same or different materials as the tracks. In some cases, it might be beneficial to construct the wiper out of a material with very high conductivity, such as a precious metal, for example platinum, gold, silver, or palladium. The same applies to the electrical connectors attached to the track ends or to the wipers that are used to establish an electrical connection to the measuring device.

The conductive tracks and wiper, as well as the electrical connectors, can be glued, press-fit, clamped, screwed, or otherwise physically attached to the dose setting components as separate items. Alternatively, the tracks, wiper and connectors could be made integral to the selected dose setting components by co-molding the tracks, wiper, or connectors when the selected dose setting component is manufactured in the first instance. Such a manufacturing process is sometimes referred to a “two-shot” molding process. Co-molding allows for economically efficient manufacturing, especially when the injection device is intended as a disposable device, meaning that the container of medicament is sealed within the device and once all of the medicament has been expelled, usually through repeated injections of the same or different doses, the injection device is then discarded. In other words, in such a disposable device there is no mechanism to remove an empty container of medicament, resetting of the piston rod, or inserting a new filled container.

As mentioned, by attaching a measuring device to the electrical connectors at the end of each track, the changing resistance of the circuit created by the wiper that bridges and connects to each track can be measured and used to determine a number of injection device parameters. The measuring device can be a non-removable component part of the injection device, especially if the device is designed as a reusable injection device. Or, in the case of disposable injection device, the measuring device is preferably a separate stand-alone part that is attached to the disposable injection device during use and then removed before the device is discarded. In this way, the measuring device would then be attached to a new, replacement disposable injection device and reused. This would be repeated for each new disposable device needed. The measuring device would have conductors configured to attach to the electrical connectors attached to either the track ends or the wiper(s). The measuring device could comprise a power supply and an application-specific integrated circuit (ASIC) connected to conductors to measure resistance in the circuit created by the wiper and two conductive tracks. The ASIC may be adapted to collect information regarding the operation of the injection device and to transform the information collected into a format recognizable to a user. A processor within the measuring device could use the measured resistance to calculate a dose setting or dose delivered. This calculated result could be transmitted to a display accessible by the user and it could be stored in memory for transmission to another device via wired or wireless connection. The processor could also include a clock function to allow it to monitor the time/date of injection, the rate of resistance change, i.e., the speed of injection, and whether the injection was halted and for how long. It is also possible that the measuring device could determine the temperature of device and hence provide an approximate temperature of the medicament at the time of injection or during non-use of the device. Temperature profiles of the injection device can be related to the effectiveness of medicaments, e.g., degree of degradation or reduced potency of the medicament.

Although not intending to be limited, several possible embodiments of this disclosure are set forth below.

A dose setting mechanism for a drug delivery device can have an outer housing, a dose sleeve configured to move axially in a distal direction relative to the outer housing during dose setting, and a potentiometer comprising a first part located on an inside surface of the outer housing and a second part located on the dose sleeve. The first part can comprise a wiper in contact with the second part that comprises a track. The dose setting mechanism can contain a wiper and a track each comprising an electrically conductive material such that an electrical resistance can be measured between the wiper and the track during relative movement between the dose sleeve and the outer housing. Electrical connectors can be included that are in electrical contact with the first and second parts of the potentiometer.

The first part can also comprise a wiper in contact with the second part that comprises two tracks longitudinal extending along the dose sleeve. In this configuration two electrical connectors can be used, where one electrical connector is in electrical contact one track and the other electrical connector is in electrical contact with the other track.

Also, the dose setting mechanism could be configured where the first part comprises two wipers each connected to an electrical contact and where the second part comprises two longitudinal tracks electrically connected to each other by a strip of conductive material, wherein one wiper is in sliding electrical contact with one track and the second wiper is in sliding electrical contact with the other track.

The dose setting mechanism of claim 1 could also include a measuring device in electrical communication with the potentiometer, such that the measuring device can monitor and determine electrical resistance between the first part and the second part. The measuring device could be configured such that it can be removed from the dose setting mechanism and reused on a second dose setting mechanism.

The dose setting mechanism could also have a second potentiometer having a first portion and a second portion, where the first portion is located on a non-rotating piston rod and the second portion is located on a piston guide. A measuring device could be in electrical communication with the first and second potentiometers, such that the measuring device can determine the relative motion and distance traveled between the dose sleeve and the outer housing and between the piston rod and the piston guide. The measuring device can use the determination of relative motion and distance traveled to determine dose accuracy.

The dose setting mechanisms described above could be incorporated into a complete drug delivery device further having a cartridge holder removably or permanently attached to the dose setting mechanism and in the case of a disposable drug delivery device having a non-replaceable cartridge contained within the cartridge holder. Further, an assembly or collection of stand-alone components is possible, for example, a drug delivery device having one of the dose setting mechanisms described above and a measuring device configured for releasable attachment to the drug delivery device such that when the measuring is attached to the drug delivery device the resistance of one or more potentiometers can be monitor and measured.

These and other aspects of, and advantages with, the present disclosures will become apparent from the following detailed description of the present disclosure and from the accompanying drawings.

### BRIEF DESCRIPTION OF DRAWINGS

In the following detailed description of the present disclosure, reference will be made to the accompanying drawings, of which

FIG. 1 is a perspective illustration of one possible complete medicament delivery device showing the conductive tracks of the present disclosure;

FIG. 2 illustrates the outside and inside of a sleeve of the device of FIG. 1;

FIGS. 3A-3D illustrates the particulars of an end ring of the housing of FIG. 1;

FIG. 4 is a perspective illustration of another possible complete medicament delivery device containing the structural components of the present disclosure;

FIG. 5 shows a perspective illustration of the device of FIG. 1 where the cap is removed allowing attachment of a pen needle to the cartridge holder;

FIG. 6 is an exploded view of the device of FIG. 4;

FIGS. 7A-7E shows conductive tracks applied to a piston rod having a non-circular cross-section;

FIG. 8 shows the piston rod of FIG. 7 being held by a piston rod guide of the device of FIG. 4;

FIG. 9 shows a perspective view of the injection device of FIG. 4 including connectors attached to two wipers in sliding contact with two conductive strips;

FIG. 10 shows a perspective view of the device of FIG. 9 having attached to the wiper connectors a measuring device;

FIG. 11 illustrates the device of FIG. 10 at a zero dose setting;

FIG. 12 illustrates the device of FIG. 10 where a dose of 30 IU has been set; and

FIG. 13 illustrates different views of the measuring device of FIG. 10.

### DETAILED DESCRIPTION

In the present application, the term “distal part/end” refers to the part/end of the device, or the parts/ends of the components or members thereof, which in accordance with the use of the device, is located the furthest away from a delivery/injection site of a patient. Correspondingly, the term “proximal part/end” refers to the part/end of the device, or the parts/ends of the members thereof, which in accordance with the use of the device is located closest to the delivery/injection site of the patient.

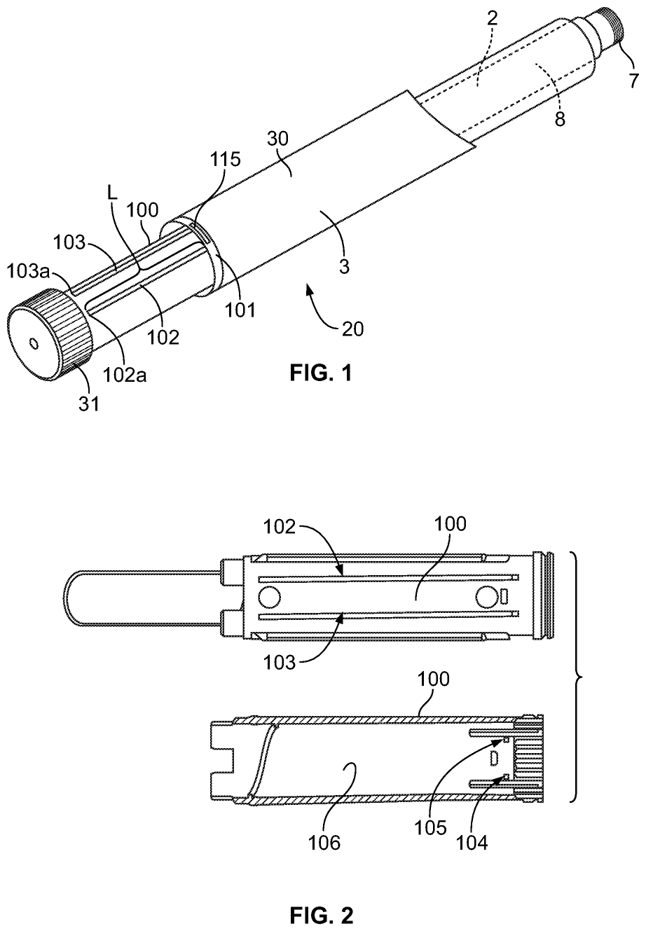

The present disclosure is applicable with a number of injection device designs. FIG. 1 illustrates one type of a pen-shaped injection design 20 having a dose setting mechanism 3 connected to a cartridge holder 2 holding cartridge 8, where the cartridge holder 2 has a needle connector 7. Device 20 has a sleeve 100 that translates in a longitudinal direction during dose setting, dose correction and dose delivery. A dose is set through rotation of dose knob 31, which causes sleeve 100 to move linearly in the distal direction. A dose is delivered by pushing on the end of the dose knob in the opposite or proximal direction. This in turn causes sleeve 100 to move linearly back into the dose setting mechanism in the proximal direction. The outer surface of sleeve 100 has two conductive tracks 102, 103 that are shown being parallel to each other and running the length of the sleeve. This is also illustrated in FIG. 2, which includes a cross sectional view of the inside surface 106 of sleeve 100 and shows two terminal points or electrical connectors 104,105 that are in electrical communication with tracks 102, 103, respectively.

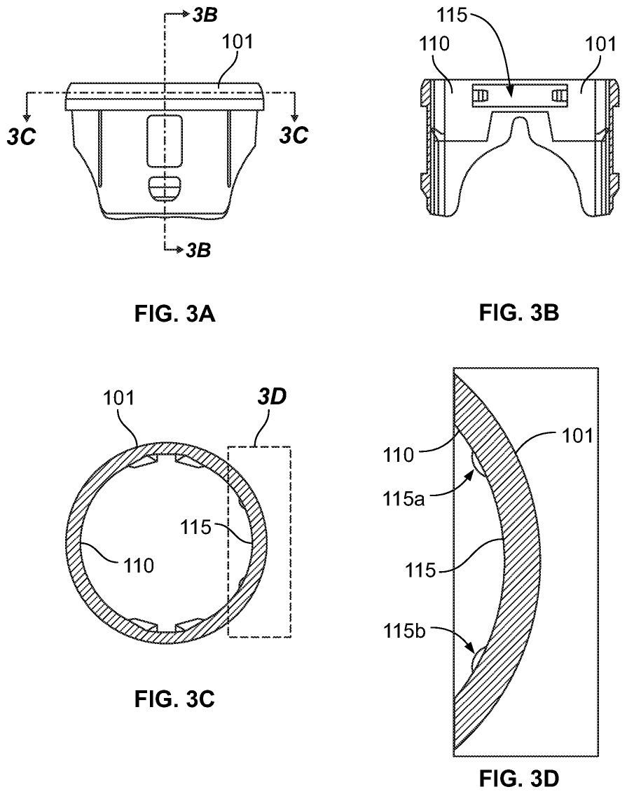

At the very distal end of housing 3 is an end ring 101 having an inside surface 110 containing wiper 115 that is in sliding contact with tracks 102, 103. End ring 101 is rotationally and linearly fixed to housing 3. Details of the end ring 101 are shown in FIG. 3. From the cross-sectional view of end rig 101 there is shown an inner surface 110 where wiper 115 is positioned such that individual wiper protrusions 115a, 115b extend radially outward so that they maintain contact with the conductive tracks as sleeve 100 moves linearly in and out of hosing 3. These two wiper protrusions are joined by a strip of conductive material. Because wiper 115 is also made of a conducting material, an electrical connection is formed between the tracks at the points where the wiper protrusions 115a, 115b, are in contact with the tracks. As illustrated in FIG. 1, the length of the tracks measured from the terminal distal ends 102a, 103a to the point of contact with the wiper is defined as L. The resistance across the distal terminal distal ends 102a, 103a is function of length L such that as L gets shorter (smaller value), for example during dose correction or dose delivery, the measured resistance is also smaller. During dose setting L is increased along with the measured resistance. A measuring device could be located within sleeve 100 with electrical connections to terminals 104 and 105 to measure the resistance of the circuit defined by the two tracks and the wiper. In this set-up the measuring device would travel in and out of the dose setting mechanism with sleeve 100. The measured resistances during dose setting, dose correction and dose delivery would be directly related to the actual amount of medicament set to be delivered and the amount of medicament actually delivered.

In an alternative embodiment of the design shown in FIGS. 1-3, the terminal distal ends 104, 105 would be connected together through a conductive strip and the conductive material between wiper protrusions 115a, 115b would be eliminated. In this design a measuring device could be connected directly to each of the wiper protrusions. This would allow the measuring device to remain in a stationary position inside the housing. Alternatively, electrical connectors could be attached to each wiper protrusion such that the connectors could be accessible from the outside of housing 3. In such a design the measuring device could then be releasably attached to the outer surface of housing 3 and would be reusable.

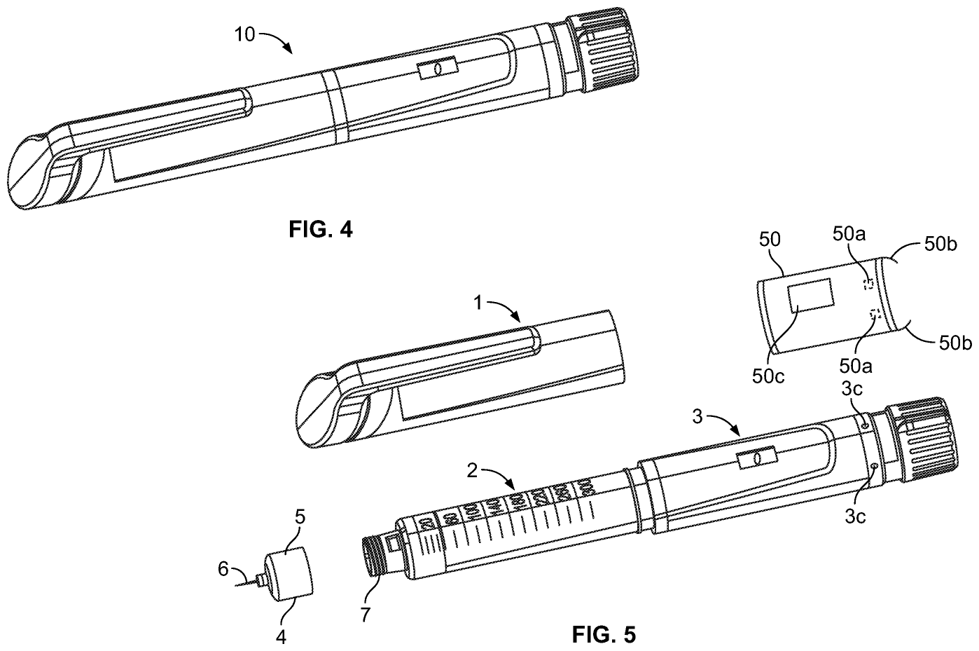

A different pen-type injector design 10 that is shown in FIG. 4 will now be described with more details as to the component parts and their operation. Like the above described design, the injection device 10 has a dose setting mechanism 30 as shown in FIG. 5. The complete injection device 10 is illustrated in the zero dose state as indicated by indicia 40 showing a zero through the window 3a of housing 3. FIG. 5 shows the device 10 with cap 1 removed to expose the cartridge holder 2 and the proximal needle connector 7. Pen needle 4 is typically attached to the needle connector 7 through a snap fit, thread, Luer-Lok, or other secure attachment with hub 5 such that a double ended needle cannula 6 can achieve a fluid communication with medicament contained in cartridge 8 positioned within cartridge holder 2. The cartridge 8 is sealed at the proximal end by septum 8a and with a sliding piston 9 at the opposite distal end.

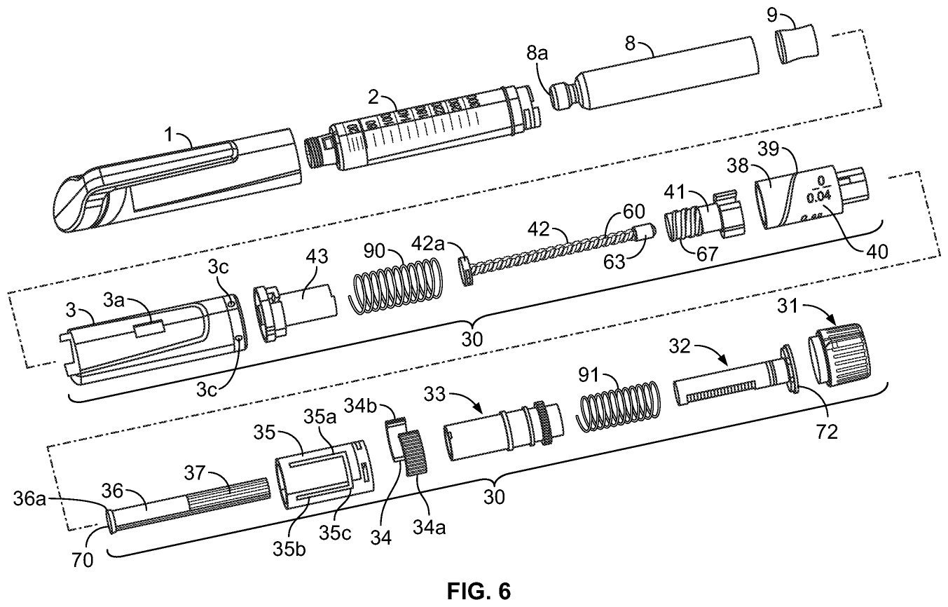

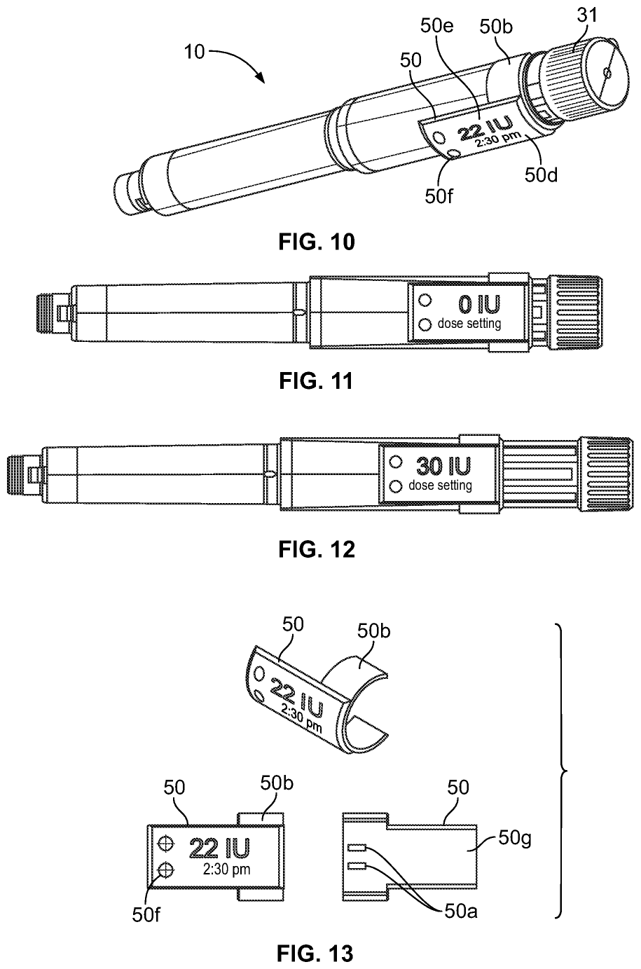

As explained above, the conductive tracks of the present invention can be located on an injector component that moves linearly during dose setting, dose delivery and dose correction. One such component is dose selector 35, which is shown in FIG. 6 as part of the dose setting mechanism 30 and has two longitudinal conductive tracks 35a and 35b positioned the outside of dose selector 35 and electrically connected to each other by conductive strip 35c. These tracks can be in a sliding contact with wiper protrusions on the inside surface of the distal end of housing 3. These wiper protrusions are fixedly attached to the inside distal end of outer housing 3 and are electrically accessible from the outside surface of housing 3. This outside access is through connectors 3c, which allows for connection to an attachable and reusable measuring device 50 (see FIGS. 5 and 10) through electrical contacts 50a on the inside surface of measuring device 50. This measuring device could be releasably attached to the injection device outer surface of housing 3 through clips 50b such that electrical connectors 50a on the inside surface 50g (see FIG. 13) will form an electrical connection with connectors 3c. A display 50c is available to present relevant information to the user, such as, for example, the time when the last injection took place 50d, and the dose amount of that last injection 50e. Clearly, other pertinent information could be displayed by the measuring device, such as battery charge level, temperature, alarm status, medicament identification information, connectivity status, etc. the measuring device could also have one or more input features 50f, such as buttons or touch screen features, for the user to press to activate the various features of the measuring device. As explained above the measuring device can measure electrical conductivity in the circuit defined by tracks 35a, 25b, and conductive strip 35c. As explained below during dose setting, dose correction, and dose delivery dose selector 35 moves linearly in and out of housing 3 and thus changes the electrical resistance the circuit.

The linear movement of the dose selector 35 is a result the outer surface that has one or more longitudinal grooves that are always engaged with longitudinal splines located on the inner surface of housing 3. This engagement prevents relative rotation between the dose selector and the housing, but allows the dose selector to move axially relative to the housing. The outer surface of the dose selector also has connecting cut-outs that permanently engage and lock with snap fits on the dose knob 31 such that the dose knob is axially fixed to the dose selector 35. These permanent snap fits allow the dose knob to rotate relative to the dose selector during both dose setting and dose cancellation.

The particular design of device 10 allows for setting of one or more of the predetermined fixed doses through the interaction of snap element 33 with dose selector 35. The rotation of the dose knob and snap element occurs during dose setting and is relative to housing 3. During the initiation of the dose delivery procedure the dose knob 31 is pressed in the proximal direction causing it and the dose selector to move axially relative to the snap element. This initial movement disengages a splined connection and causes engagement of a different spline connection which prevents the doe knob from rotating relative to the housing 3 during dose delivery. The initial movement of the dose selector proximally does not cause movement of the piston rod 42. As such, this initial movement of the dose selector 35, and the conductive tracks, would result in a measured change (decrease) of resistance, however, this would not be indicative of an amount of dose delivered. To compensate for this “false” dose delivery based on the resistance reading, a switch mechanism could be used to signal the measuring device that the initial movement of the dose selector was completed and then to start recording the resistance changes during the actual dose delivery. Since the initial proximal movement of the dose selector is generally always the same regardless of the dose set, then the measuring device could be programed to ignore or subtract out the small delta resistance difference caused by the initial movement that occurs before the piston rod actually begins to move proximally.

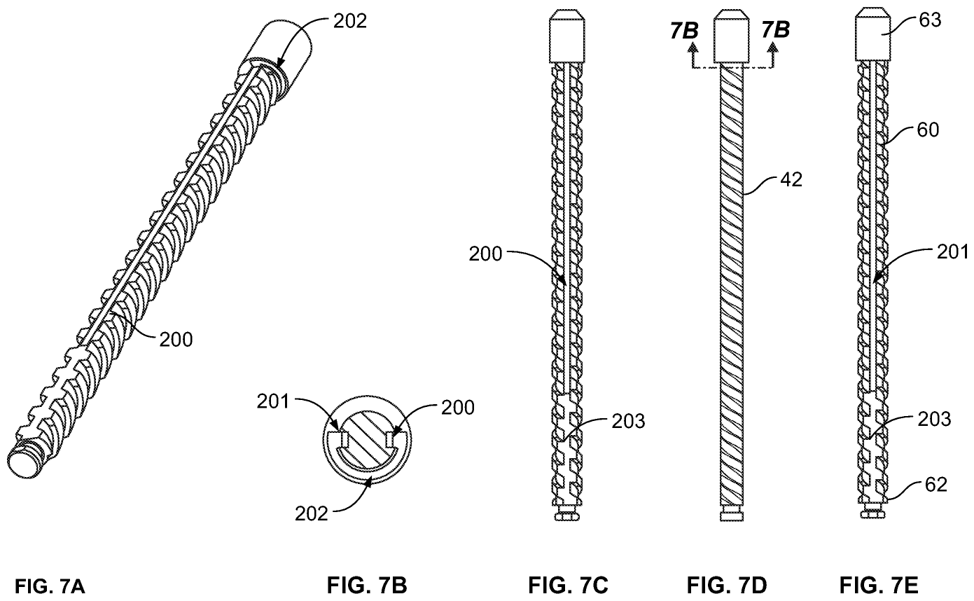

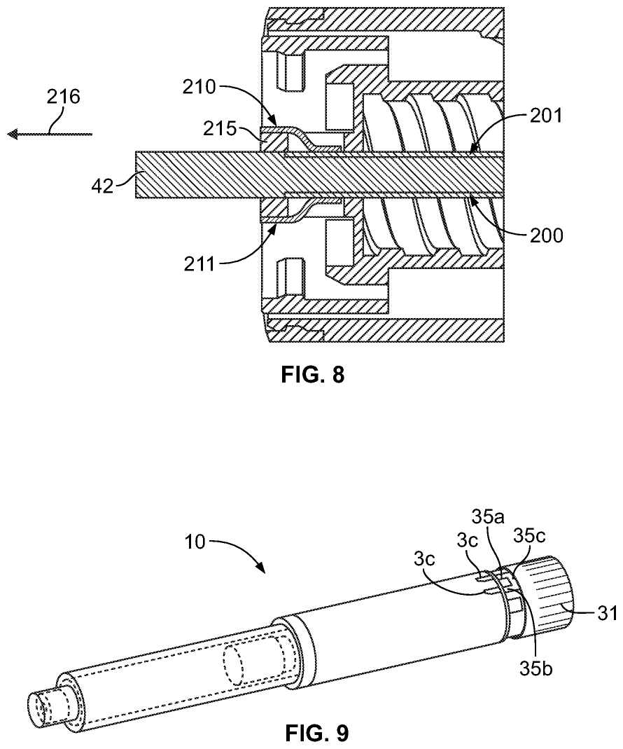

Part of the dose setting mechanism of most pen-type injectors, including device 10, is a piston rod 42 as illustrated in FIGS. 6-8. In those device designs where the piston rod does not rotate during dose delivery, conductive tracks maybe applied to the outer surface of the piston rod as illustrated in FIGS. 7 and 8. Such piston rods usually have a non-circular cross-section and have two flat surfaces 203 that are designed to prevent the piston rod from rotating, but allowing it to move linearly in the proximal direction. With such piston rod designs, conductive tracks 200, 201 can be applied to or made integral with the flat sides 203, for example by co-molding. The two tracks 200, 201 can be electrically connected through conductor strip 202. Two wipers 210, 211 can be incorporated in a piston guide 215 that is linearly fixed to the housing such that one wiper is in sliding electrical contact with one track and the other wiper in electrical contact with the other track as illustrated in FIG. 8. As the piston rod moves proximally as designated by arrow 216, the length of the two tracks 200, 201 decreases, which causes a decrease in the resistance of the circuit defined by tracks 200, 201, conductor strip 202. A measuring device could be electrically connected to the wipers 210, 210 so that the resistance of the circuit could be measured. Such a piston rod design could be used alone or in conjunction with the above-mentioned circuit defined by tracks 35a, 35b and conductive strip 35c. Such a combination of linear potentiometers would allow both the piston rod movement and the dose selector movement to be evaluated separately during dose delivery and then the collected data could cross-check to evaluate the accuracy of the actual dose being delivered.

Returning to the specifics of the dose setting mechanism 30 of device 10, a nut 36 and a clutch 32 are permanently splined to each other during assembly of the dose setting mechanism through a splined connection. The splined connection ensures that clutch 32 and nut 36 are always rotationally fixed to each other during both dose setting and dose delivery. This splined connection also allows the clutch and the nut to move axially relative to each other. The sliding connection is necessary to compensate for the difference in the pitch of the thread between nut and the outer surface of the piston rod and the pitch of the thread between dose sleeve and body. The thread between driver and piston guide has basically the same pitch as the thread between piston rod and nut.

The proximal end of nut 36 has internal threads 70 that match threads 60 of piston rod 42. The distal end of clutch 32 is configured as a dose button 72 and is permanently attached to distal end of the dose knob 31 through engagement of connectors, which may also include snap locks, an adhesive and/or a sonic weld. This connection ensures that the clutch is both rotationally and axially fixed to the dose knob during both dose setting and dose delivery.

In addition to threads 60 on the outer surface of the piston rod 42, there is also included two longitudinal flats 203, as described above, that give piston rod 42 a non-circular cross section. At the terminal proximal end is connector 62, shown as a snap fit, that connects with a disc or foot 42a. At the distal end of piston rod 42 is a last dose feature of the dose setting mechanism, illustrated as an enlarged section 63. This enlarged section 63 is designed to stop the rotation of nut 36 about threads 60 when the amount of medicament remaining in the cartridge 8 is less than the next highest predetermined dose setting. In other words, if the user tries to set one of the predetermined fixed dose settings that exceeds the amount of medicament remaining in the cartridge, then the enlarged section 63 will act as a hard stop preventing the nut from further rotation along threads 60 as the user attempts to reach the desired predetermined fixed dose setting.

The piston rod 42 is held in a non-rotational state relative to housing 3 during both dose setting and dose delivery because it is arranged within the non-circular pass through hole 215 in the center of piston rod guide 43 (see FIG. 8). The piston rod guide is both rotationally and axially fixed to housing 3. This fixation can be achieved when the piston rod guide is a separate component from the housing 3 as illustrated in the figures or the piston rod guide could be made integral with the housing. Piston rod guide 43 also engages the proximal end of a rotational biasing member, shown as torsion spring 90, the function of which will be explained below. This connection of the rotational biasing member to the piston rod guide anchors one end in a rotational fixed position relative to the housing.

The distal end of the rotational biasing member, for example torsion spring 90, is connected to the driver 41. Driver 41 is connected and rotationally fixed with the inner surface of dose sleeve 38 through a splined connection on the distal outer surface of the driver. On the proximal end of driver 41 on the outer surface are threads 67 that are engaged with matching threads on the inner distal surface of the piston rod guide 43. The thread between driver and piston guide has a significantly different pitch than the thread between dose sleeve and housing. The nut and the driver rotate together both during dose setting and dose cancellation and, as such, they perform essentially the same axial movement. However, this movement is independent from each other, i.e., the nut is turned by the clutch and performs an axial movement due to the thread to the piston rod, while the driver is rotated by the dose sleeve and performs an axial movement due to the thread to the piston guide. The driver is rotating during injection also, and so it actively moves in the proximal direction during injection. But, the nut does not rotate during injection and as such does not perform an active axial movement. The nut is only moving in proximal direction during injection because it is being pushed axially by the driver. The rotating driver pushing the non-rotating nut causes the injection because the piston rod is pushed forward due to the threaded engagement with the nut.

If, for example, the thread of the nut had a higher pitch than the thread of the driver, the nut could not freely move in the distal direction during dose setting because it would be hindered by the slower moving driver. As such, this would cause drug to be expelled during dose setting. Alternatively, if the thread of the nut had a significantly lower pitch than the thread of the driver, the driver would move away from the nut during dose setting and the driver would not push the nut at the beginning of the injection already, but would do so only after the gap is closed. Accordingly, it is preferred that the pitch of the thread on the driver is equal or a slightly higher than the pitch of the thread on the nut. And, the thread between the dose sleeve and the housing has a higher pitch than that of the nut and piston rod. This is desirable because it yields a mechanical advantage that makes the dose delivery process easier for the user. For example, when pushing the knob a distance of 15 mm, the piston rod only moves by 4.1 mm. This results in a gearing ratio of about 3.6:1. A lower gearing ratio would result increase the force the user needs to complete the injection.

Because the torsion spring is attached to the driver 41 and the driver is rotationally fixed to the dose sleeve 38, then rotation of the dose sleeve in a first direction during dose setting will wind the torsion spring such that it exerts a counter rotational force on the dose sleeve in an opposite second direction. This counter rotational force biases the dose sleeve to rotate in a dose canceling direction.

The function of the complete injection device 10 and the dose setting mechanism 30 according to this disclosure will now be described. Injection device 10 is provided to a user with or without the cartridge 8 of medicament positioned within the cartridge holder 2. If the injection device 10 is configured as a reusable device, then cartridge holder 2 is connected to housing 3 of the dose setting mechanism 30 in a releasable and reusable manner. This allows the user to replace the cartridge with a new full cartridge when all the medicament is expelled or injected from the cartridge. If the device is configured as a disposable injection device, then the cartridge of medicament is not replaceable because the connection between the cartridge holder 2 and the housing 3 is permanent. Only through breaking or deformation of this connection can the cartridge be removed from the injection device. Such a disposable device is designed to be thrown out once the medicament has been expelled from the cartridge.

The user first removes the cap 1 from the device and installs an appropriate pen needle 4 to the cartridge holder 2 using connector 7. If the device is not pre-primed during the device assembly or does not have an automatic or forced priming feature, then the user will need to manually prime the device as follows. The dose knob 31 is rotated such that a first dose stop is reached, which corresponds to a predetermined small fixed dose of medicament.

The injection device 10 of this disclosure can also have a so-called forced or automatic priming feature. Prior to using the dose setting mechanism, i.e., before a user could dial one of the predetermined fixed dose setting, a sliding lock would necessarily need to push in the proximal direction such that is moves distally relative to the dose knob. This axial movement forms an irreversible locking relationship between the dose knob and the distal end of the clutch. This locking relationship also causes the dose knob and clutch to be rotationally fixed to each other. Before the sliding lock is engaged with the clutch, the clutch can be rotated, which also causes rotation of the nut, to cause the piston rod 42 to move axially relative to the housing. The clutch is rotated until a visual observation and/or tactile notification indicates that the foot 42a located on the piston rod 42 is in firm abutment with distal facing surface of the sliding piston 9. This abutment between the foot and the sliding piston will ensure that an accurate dialed dose will be delivered out of the needle cannula. This rotation of the clutch is preferably performed during the assembly of the injection device and likewise after ensuring abutment of the foot with the sliding piston 9, the manufacturing process would cause the sliding lock to be pushed to the final, locked position.

Returning to the priming procedure, once the priming stop is reached, the user may need to cancel the priming procedure and can do so by using the dose canceling procedure. This cancellation procedure also applies to any dose setting. Dose cancellation is accomplished by turning the dose knob in the opposite direction and will generate a notification that can be the same or different as the dose setting notification and/or dose delivery notification. Because the snap element 33 is rotationally fixed to the dose sleeve 38, and the dose sleeve is threaded engaged to the inner surface of housing 3, rotation of the dose knob during dose setting and dose cancellation causes relative rotation between the dose sleeve and the housing. The threaded connection between the housing and the dose sleeve causes the dose sleeve, snap element, clutch, and dose knob to translate axially as the dose knob is rotated. During dose cancellation, these components rotate and translate axially in the opposite or proximal direction.

Rotation of the dose knob also causes rotation of nut 36 about threads 60 on the outer surface of piston rod 42, which does not rotate and remains axially fixed relative to the housing 3 because of relative pitch differences in the threaded parts as explained above. The rotation of the nut relative to the stationary piston rod, which is supported by its contact with the sliding piston, causes the nut to translate or climb up the piston rod in the distal direction. A reverse rotation during dose cancellation causes the nut to translate in the reverse direction relative to piston rod. The distance traveled by the nut to achieve the desired dose setting is directly proportional to an amount of medicament that would be expelled if the dose delivery procedure were initiated and completed. Because the pitch of the threaded connection between the dose sleeve and the housing is greater than pitch of the threads on the nut, the dose sleeve, snap element, clutch and dose knob will travel a greater axial distance than the nut as it climbs up or down the piston rod. The difference in axial movement would normally bind the dose setting mechanism, but does not do so because the difference in pitch is compensated for by the sliding splined connection between the nut and the clutch, thus allowing the clutch to travel axially a greater distance longitudinally than the nut. During injection, the clutch pushes on the snap element and as such on the dose sleeve. This axial force causes the dose sleeve to turn due to the thread to the body. The dose sleeve will only start to turn when it is pushed, if the pitch of the thread is high enough. If the pitch is too low the pushing will not cause rotation because the low pitch thread becomes what is called a “self-locking thread.”

Rotation of the dose knob also causes rotation of the driver because of the splined rotationally fixed connection to the dose sleeve. Since the torsion spring 90 is fixed at one end to the driver and at the other end to the piston rod guide, which in turn is fixed axially and rotationally to the housing, the torsion spring is wound up increasing in tension during dose setting. As mentioned, the torque of the tension spring exerts a counter rotational force on the dose sleeve. Preferably during assembly of the dose setting mechanism, the torsion spring is pre-tensioned so that even at the zero dose condition the torsion spring exerts a counter rotational force on the dose sleeve. The counter rotation force provides a first fail-safe feature of the dose setting mechanism. This first fail-safe mechanism prevents a user from setting a dose that is not one of the finite set of predetermined dose settings. In other words, if a user is rotating the dose knob such that it is between two dose stops, or between the zero dose hard stop and a first dose stop or a priming stop, and the user releases the dose knob, the counter rotational force of the torsion spring will return the protrusion to the last engaged dose stop or to the zero dose hard stop. Additionally, during a dose cancellation procedure the counter rotational force will assist the user in rotating the dose knob back down to the next lower fixed dose setting or possibly all the way back to the zero dose setting.

During dose setting, the dose knob 31 translates out and away from the distal end of housing 3. As the dose sleeve 38 rotates and translates, the progress of the dose setting (or dose cancellation) is observed in window 3a of housing 3 as the printed indicia 40 on the dose sleeve moves past the open window. This movement of the dose sleeve also causes linear movement of the dose selector 35, which can be measured by measuring the electrical conductivity of the circuit defined by tracks 35a, 35b and conductor strip 35c. When a desired predetermined dose setting is reached the indicia for that dose will appear in the window. At this point the injection device 10 is ready for a priming procedure or, if already primed, the delivery of the medicament to an injection site. In either the case, the user will push on the dose knob in the proximal direction until the zero dose hard stop is reached and a zero dose indicia is observed in the window. During a priming step the user will observe whether medicament is expelled out of the cannula 6 of pen needle 4. If no medicament is expelled this means the piston foot 42a is not in abutment with the distal surface of sliding piston 9. The priming step is then repeated until medicament is observed exiting the cannula.

The dose setting mechanism of the present disclosure can also have a maximum dose hard stop feature that prevents a user from setting a dose greater than the highest predetermined dose setting.

Once the dose setting mechanism is primed, the user then selects and sets a desired fixed dose by repeating the same steps used for priming except that the dose knob will be rotated past the priming stop until the appropriate dose stop is and the desired dose value appears in the window 3a. In some cases, it is preferred to have no indicia show in the window when dialing between predetermined dose settings, while in other cases it is desirable to show indicia in the window that is indicative of a non-settable dose position between the fixed dose settings.

Once one of the predetermined dose settings has been dialed on the dose setting mechanism, the user can then exert an axial force in the proximal direction to initiate the dose delivery procedure. The axial force exerted by the user overcomes the distally directed force exerted by the second biasing member 91 causing the dose knob 31, clutch 32 and dose selector 35 to move axially in the proximal direction relative to the snap element 33 and housing 3. This initial movement rotationally fixes the clutch and dose knob to the housing through the splined connection between the floating spline 34 and splines inside dose selector 35. The splined connection between the dose selector and floating spline 34 remains engaged during dose setting and during dose delivery even though the dose selector 35 moves axially with the dose knob 31 and relative to the floating spline 34.

As the user maintains the axial force on both the dose knob 31 and the dose button 72 during the continuation of the dose delivery procedure, the clutch 32 will abut the distal end of the snap element causing it to move axially in the proximal direction. The clutch pushes on the snap element. The snap element is fixed to the dose sleeve, so the clutch pushes on the dose sleeve. As the dose sleeve has a thread with a sufficiently high pitch relative to the body, the axial force on the dose sleeve will cause the dose sleeve and as such the snap element to turn relative to the body, and by turning relative to the body it moves in the proximal direction. The dose selector slides into the housing but does not rotate relative to the housing 3 due to the splined engagement with the housing. Again, the linear movement of the dose selector in the proximal direction can be determined by measuring the electrical conductivity of the circuit formed by tracks 35a, 35b and strip 35c. The rotation of the dose sleeve 38 also causes rotation of the driver 41 into the threaded connection with piston rod guide 43, which drives the piston rod proximally and results in a concurrent de-tensioning of torsion spring 90. The driver does not directly drive the piston rod. As the driver rotates, the driver moves in the proximal direction and pushes the nut forwards. As the nut doesn't turn, the driver pushes the nut and the piston rod forward.

The nut 36 does not rotate during dose delivery because of the rotationally fixed relationship with clutch 32 that is rotationally fixed to the housing through rotationally fixed relationship of the dose knob, floating spline and the housing. The nut therefore can only move axially carrying the piston rod 42 with it because the piston rod is prevented from rotating by the non-circular opening 64 engaged with the flats 203 on the piston rod. The piston rod is moved axially the same distance that the nut originally translated relative to the piston rod during dose setting. Again, this movement of the piston rod can be determined through the electrical conductivity measurement described above. This axial movement without rotation is caused by the rotational and axial movement of the proximal end of the driver in abutment with a flange 36a on nut 36. Axial movement of the piston rod causes the sliding piston 9 to also move axially relative to the inside walls of the stationary cartridge 8 forcing an amount of medicament out of the needle cannula 6 that is equivalent to the predetermined fixed dose that was set during the dose setting procedure.

If the user stops or halts the dose delivery procedure by removing the axial force on the dose knob a fail-safe mechanism is activated. Removal of the axial force causes the compression spring 91 to bias the dose knob in the distal direction. If the user halts the dose delivery between two predetermined fixed dose settings, then the dose knob and the axially fixed dose selector will both be prevented from moving proximally because of a projecting rib inside the dose selector that will stop the axially movement of dose selector and dose knob. Without this projecting rib, the dose selector would move distally such that the dose knob would re-engage with the snap element, thus placing the dose knob, clutch and nut back into rotational engagement with the snap element. The torque exerted on the snap element through the driver would then counter rotate the nut, thus reducing the set dose by an unknown amount. This counter rotation would continue until the next lowest predetermined fixed dose setting is reached, where the corresponding dose stop would stop the counter rotation. Therefore, a resumption of the halted dose delivery procedure will continue without any unknown decrease in the set dose, thus allowing the originally set predetermined dose to be delivered. A halted dose delivery could be determined using the electrical circuits described above because the measuring device would sense a rate change of resistance. Likewise, a halted dose delivery could be determined and recorded by using a clock function of the measuring device that would sense no change of resistance over a period of time for the injection corresponding to the halted injection.

It is to be understood that the embodiments described above and shown in the drawings are to be regarded only as non-limiting examples of the possible designs of the safety assembly and such designs may be modified in many ways within the scope of the patent claims.

What is claimed is:

1. A dose setting mechanism for a drug delivery device comprising:
an outer housing;
a dose sleeve configured to move axially in a distal direction relative to the outer housing during dose setting; and
a potentiometer comprising a first part located on an inside surface of the outer housing and a second part located on the dose sleeve.

2. The dose setting mechanism of claim 1 where the first part comprises a wiper in contact with the second part that comprises a track.

3. The dose setting mechanism of claim 2 where the wiper and track comprise an electrically conductive material such that an electrical resistance can be measured between the wiper and the track during relative movement between the dose sleeve and the outer housing.

4. The dose setting mechanism of claim 3 further comprises electrical connectors in electrical contact with the first and second parts of the potentiometer.

5. The dose setting mechanism of claim 1 where the first part comprises a wiper in contact with the second part that comprises two tracks longitudinal extending along the dose sleeve.

6. The dose setting mechanism of claim 5 further comprising two electrical connectors, where one electrical connector is in electrical contact one track and the other electrical connector is in electrical contact with the other track.

7. The dose setting mechanism of claim 1 where the first part comprises two wipers each connected to an electrical contact and where the second part comprises two longitudinal tracks electrically connected to each other by a strip of conductive material,
wherein one wiper is in sliding electrical contact with one track and the second wiper is in sliding electrical contact with the other track.

8. The dose setting mechanism of claim 1 further comprising a measuring device in electrical communication with the potentiometer, such that the measuring device can monitor and determine electrical resistance between the first part and the second part.

9. The dose setting mechanism of claim 8 where the measuring device is configured such that it can be removed from the dose setting mechanism and reused on a second dose setting mechanism.

10. The dose setting mechanism of claim 1 further comprising a second potentiometer having a first portion and a second portion, where the first portion is located on a non-rotating piston rod and the second portion is located on a piston guide.

11. The dose setting mechanism of claim 10 further comprising a measuring device in electrical communication with the first and second potentiometers, such that the measuring device can determine the relative motion and distance traveled between the dose sleeve and the outer housing and between the piston rod and the piston guide.

12. The dose setting mechanism of claim 11 where the determination of relative motion and distance traveled is processed by the measuring device to determine dose accuracy.

13. A drug delivery device comprising;
a dose setting mechanism as claimed in claim 4;
a cartridge holder permanently attached to the dose setting mechanism; and
a non-replaceable cartridge contained within the cartridge holder.

14. An assembly comprising:
a drug delivery device comprising a dose setting mechanism as claimed in claim 4; and
a measuring device configured for releasable attachment to the drug delivery device such that when the measuring is attached to the drug delivery device the relative resistance between the first and second parts can be monitor and measured.

15. The assembly of claim 14 wherein the dose setting mechanism further comprises a second potentiometer as claimed in claim 10, where the measuring device monitors and measures resistance of the second potentiometer.
